(19) United States
(12) Patent Application Publication
(10) Pub. No.: US 2026/0341412 A1
(43) Pub. Date: Oct. 29, 2026

(54) MANAGING VARIABILITY OF GENERATIVE ARTIFICIAL INTELLIGENCE (AI) MODEL OUTPUTS USING STRUCTURED REPRESENTATIONS

(71) Applicant: Dell Products L.P., Round Rock, TX (US)

(72) Inventors: OFIR EZRIELEV, Be'er Sheva (IL); HANNA YEHUDA, Acton, MA (US); ANNE-MARIE MCREYNOLDS, San Jose, CA (US)

(21) Appl. No.: 19/191,524

(22) Filed: Apr. 28, 2025

(51) Int. Cl. G16B 40/00; G16B 45/00

(52) U.S. Cl. CPC G16B 40/00; G16B 45/00

(57) ABSTRACT

Methods and systems for providing computer-implemented services using generative AI models are disclosed. To do so, a prompt may be obtained for a generative AI model of the generative AI models. The prompt may be iteratively perturbated to obtain perturbated prompts. The perturbated prompts and the generative AI model may be used to obtain perturbated outputs. Sets of corresponding information elements of the perturbated outputs may be identified, and a phylogenetic representation may be identified for a set of the sets of corresponding information elements. Variability information may be obtained for elements of the phylogenetic representation. A portion of the computer-implemented services may be provided to a downstream consumer using at least the variability information.

### FIELD

Embodiments disclosed herein relate generally to providing computer-implemented services using generative artificial intelligence (AI) models. More particularly, embodiments disclosed herein relate to managing variability of generative AI model outputs using structured representations.

### BACKGROUND

Computing devices may provide computer-implemented services. The computer-implemented services may be used by users of the computing devices and/or devices operably connected to the computing devices. The computer-implemented services may be performed with hardware components such as processors, memory modules, storage devices, and communication devices. The operation of these components and the components of other devices may impact the performance of the computer-implemented services.

### BRIEF DESCRIPTION OF THE DRAWINGS

Embodiments disclosed herein are illustrated by way of example and not limitation in the figures of the accompanying drawings in which like references indicate similar elements.

FIG. 1 shows a diagram illustrating a system in accordance with an embodiment.

FIGS. 2A-2B show flow diagrams illustrating data flows in accordance with an embodiment.

FIGS. 2C-2G show examples of a graphical user interface (GUI) and corresponding information elements in accordance with an embodiment.

FIG. 3 shows a flow diagram illustrating a method for providing computer-implemented services using generative AI models in accordance with an embodiment.

FIG. 4 shows a block diagram illustrating a data processing system in accordance with an embodiment.

### DETAILED DESCRIPTION

Various embodiments will be described with reference to details discussed below, and the accompanying drawings will illustrate the various embodiments. The following description and drawings are illustrative and are not to be construed as limiting. Numerous specific details are described to provide a thorough understanding of various embodiments. However, in certain instances, well-known or conventional details are not described in order to provide a concise discussion of embodiments disclosed herein.

Reference in the specification to “one embodiment” or “an embodiment” means that a particular feature, structure, or characteristic described in conjunction with the embodiment can be included in at least one embodiment. The appearances of the phrases “in one embodiment” and “an embodiment” in various places in the specification do not necessarily all refer to the same embodiment.

References to an “operable connection” or “operably connected” means that a particular device is able to communicate with one or more other devices. The devices themselves may be directly connected to one another or may be indirectly connected to one another through any number of intermediary devices, such as in a network topology.

In general, embodiments disclosed herein relate to methods and systems for providing computer-implemented services using generative AI models. The generative AI models may include large language models (LLMs) that may generate outputs when provided with prompts. The outputs may be used, at least in part, to provide the computer-implemented services. For example, the outputs may be provided to a downstream consumer and the downstream consumer may use the outputs to make decisions. However, a usefulness (e.g., reliability, usability) of the outputs to the downstream consumer may be reduced if the downstream consumer does not understand why a particular output was generated.

For example, a downstream consumer (e.g., a client, a customer) may rely on an output from a generative AI model to manage a computer system. The computer system may access and/or store sensitive data such as private medical information, private financial information, and/or other data subject to by various regulatory standards (e.g., general data protection regulation (GDPR), health insurance portability and accountability act (HIPPA)).

However, without an understanding of a decision-making process by the generative AI model while generating the output, the downstream consumer may consider the output untrustworthy and may be unwilling to use the output to manage the computer system considering a risk of exposure and/or loss of the sensitive data. For example, slight variations of a prompt provided to the generative AI model may yield highly variable outputs. The variability of the outputs may be used to analyze the output prior to making a decision. For example, the variability of the outputs may indicate whether the generative AI model and/or the outputs are stable with respect to the prompt (e.g., whether an output of the generative AI model is trustworthy for direct use in decision-making).

However, a portion of the variability in the outputs may be due to different information elements (e.g., words, phrases) of the outputs that may be used to convey similar or related concepts. Therefore, to analyze such variability, a structured representation for the information elements present in the outputs may be obtained. The structured representation may be used to analyze portions of the output (e.g., information elements present in the outputs) with respect to their relationships to one another, and to facilitate visualization of variability information for the portions of the output.

Thus, to improve a usefulness of outputs from the generative AI models, the downstream consumers may be provided with variability information for the outputs along with the outputs generated by the generative AI model. The variability information may provide the downstream consumers with insight into decision-making processes of the generative AI model and insight into a manner of proceeding with use of the outputs. For example, based on the variability information, the downstream consumer may confirm trustworthiness of the output, make adjustments to the prompt, and/or analyze portions of the output to improve decision-making processes that rely on the output.

The variability information may be provided to the downstream consumers via a graphical user interface (GUI) populated with a structured representation of information elements of the outputs (e.g., a phylogenetic representation, a philological representation) to as a visual means of analysis by the downstream consumers.

By doing so, downstream consumers of outputs generated using generative AI models may be more likely to make timely decisions regarding use of the outputs of the generative AI model and therefore more likely to be provided with desired computer-implemented services.

In an embodiment, a method for providing computer-implemented services using generative AI models is provided. The method may include: obtaining a prompt for a generative AI model of the generative AI models; iteratively perturbating the prompt to obtain perturbated prompts; using the generative AI model and the perturbated prompts to obtain perturbated outputs; identifying, using an alignment algorithm, sets of corresponding information elements of the perturbated outputs; obtaining, for a set of the sets of corresponding information elements, a phylogenetic representation for the set; obtaining variability information for elements of the phylogenetic representation; and, providing a portion of the computer-implemented services to a downstream consumer using at least the variability information.

Identifying the sets of corresponding information elements may include, for a first perturbated output of the perturbated outputs: obtaining a first sequence of tokens corresponding to the first perturbated output; aligning the first sequence of tokens to other sequences of tokens corresponding to other perturbated outputs of the perturbated outputs to obtain a first set of aligned sequences; and, treating corresponding tokens of the first set of aligned sequences as a first set of the sets of corresponding information elements.

Aligning the first sequence of the tokens may include shifting, using a mathematical formulation, the other sequences relative to the first sequence to increase alignment between tokens of the first sequence and tokens of the other sequences.

A token of the tokens may include at least one type of token selected from a list of types of tokens consisting of: a word, a letter, a punctuation mark, and a phrase.

Obtaining the variability information for the set may include obtaining a characterization for a first element of the elements of the phylogenetic representation, the characterization being based on at least one selected from a list consisting of: a number of occurrences of a first information element corresponding to the first element in the perturbated outputs, and a relative frequency of occurrences of the first information element with respect to occurrences of a second information element corresponding to a second element of the elements of the phylogenetic representation in the perturbated outputs.

Obtaining the phylogenetic representation for the set may include: identifying unique information elements of the set; obtaining a first embedding of a first unique information element of the unique information elements; and, performing a phylogenetic analysis process using at least the first embedding to obtain at least a portion of the phylogenetic representation.

The phylogenetic representation may include a phylogenetic tree, the phylogenetic tree including nodes representing a portion of information elements of the perturbated outputs, and the nodes being connected by branches indicating relationships between the portion of information elements.

The method may further include obtaining an enhanced phylogenetic representation based on the phylogenetic representation and using at least portions of the variability information corresponding to each of the nodes.

The enhanced phylogenetic representation may include a phylogram, the phylogram including at least one node representing an information element that does not correspond to any of the information elements represented by nodes of the phylogenetic tree.

Providing the portion of the computer-implemented services may include populating, using the phylogenetic representation and/or the enhanced phylogenetic representation, a graphical user interface (GUI) to obtain a populated GUI, the populated GUI being viewable by the downstream consumer, and the phylogenetic representation and/or the enhanced phylogenetic representation being rendered based on the variability information to facilitate discrimination of portions of the phylogenetic representation and/or portions of the enhanced phylogenetic representation for an objective of a user of the populated GUI.

The phylogenetic representation and/or the enhanced phylogenetic representation may be rendered using a rendering schema, the rendering schema specifying a branch style and/or a node style based on the variability information.

The generative AI model may be a large language model (LLM).

In an embodiment, a non-transitory media is provided. The non-transitory media may include instructions that when executed by a processor cause the computer-implemented method to be performed.

In an embodiment, a system is provided. The system may include the non-transitory media and a processor, and may perform the computer-implemented method when the computer instructions are executed by the processor.

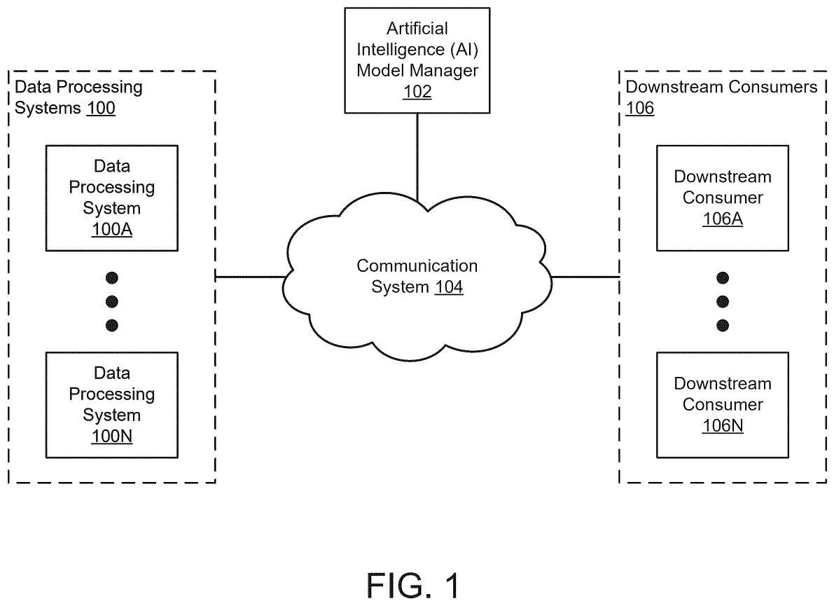

Turning to FIG. 1, a block diagram illustrating a system in accordance with an embodiment is shown. The system shown in FIG. 1 may provide for management of data processing systems that may provide, at least in part, computer-implemented services (e.g., to users of the system and/or devices operably connected to the system).

The computer-implemented services may include any type and quantity of computer-implemented services. The computer-implemented services may include, for example, database services, data processing services, electronic communication services, inference generation services, and/or any other services that may be provided using one or more computing devices. The computer-implemented services may be provided by, for example, data processing systems 100, AI model manager 102, downstream consumers 106, and/or any other type of devices (not shown in FIG. 1). Other types of computer-implemented services may be provided by the system shown in FIG. 1 without departing from embodiments disclosed herein.

The computer-implemented services may be provided, at least in part, using generative AI models and/or outputs (e.g., inferences) obtained using the generative AI models. To do so, data processing systems 100 and/or AI model manager 102 may host any number of generative AI models.

To obtain the outputs, the generative AI models may be trained, using training data, to generate the outputs when provided with prompts (e.g., ingest data). The generative AI models may include large language models (LLMs) and, therefore, the outputs may include new instances of data created by the generative AI models based on learned associations from and/or an understanding of the training data. For example, the generative AI models may be trained using unstructured data, such as stories, essays, audio transcription, video description, and/or other types of human interpretable text, to generate outputs of the same. The outputs may be provided to downstream consumers 106 (e.g., client devices, data processing systems that manage computer systems and/or production systems) as part of providing the computer-implemented services.

For example, the outputs may be provided to downstream consumers 106 as part of: (i) customer service requests (e.g., troubleshooting computer systems), (ii) data management processes (e.g., healthcare data, financial data), (iii) production system management processes (e.g., industrial applications), and/or (iv) other processes or applications.

For the outputs to be reliable for use (e.g., trustworthy) by downstream consumers 106, the outputs may be required to meet certain expectations. For example, downstream consumers 106 may expect the generative AI models and the outputs to comply with data privacy regulations (e.g., GDPR, HIPPA), may desire to mitigate unintended bias in outputs (e.g., when using the responses to make decisions based on financial data of consumers), and/or may otherwise wish to understand, audit, and/or modify operation of the generative AI models. However, due to a lack of transparency in decision-making by the generative AI models, downstream consumers 106 may be unable to determine whether the generative AI models and/or the outputs meet their expectations; therefore, the outputs may be unreliable for use by downstream consumers 106.

In general, embodiments disclosed herein may provide methods, systems, and/or devices for providing downstream consumers of generative AI model outputs with information regarding usability of the outputs. The information may indicate (e.g., in human-interpretable text and/or graphics) stability of portions of the output, which may provide the downstream consumers with information usable to determine manners of use of the outputs.

For example, a generative AI model may be sensitive to slight variations of a prompt. The slight variations may include removal of portions of the prompt and/or different phrasings of a prompt, such as using different words with similar (e.g., same, related) meanings. The slight variations of the prompt may result in a high degree of variability of corresponding outputs, indicating poor stability of the generative AI model and/or its output. Therefore, measures of variability of the outputs may be provided to downstream consumers 106 for assessment of the output.

To do so, a prompt for a generative AI model may be obtained. The prompt may include information elements such as words and/or phrases. The prompt may be iteratively perturbated (e.g., some information elements of the prompt may be removed and/or other information elements of the prompt may be replaced with replacement information elements) to obtain perturbated prompts. For example, the perturbated prompts may be provided to the generative AI model as ingest data, and each of the perturbated outputs may be generated in response to each of the perturbated prompts.

Using an alignment algorithm, sets of corresponding information elements of the perturbated outputs may be identified. For example, the alignment algorithm may be used to identify corresponding tokens (e.g., words, phrases, letters, punctuation) across aligned sequences of tokens. A portion of the corresponding tokens may be treated as a set of the sets of corresponding information elements. A phylogenetic representation for the set of corresponding information elements may be defined to provide a structured representation of the variance among portions of the perturbated outputs. For example, the phylogenetic representation may be defined using elements corresponding to information elements, and relationships (e.g., lexical relationships) between portions of the elements.

The elements of the phylogenetic representation may be characterized to obtain variability information for the set (e.g., for each set) of corresponding information elements. The variability information for the set may include (statistical) characterizations for elements of the phylogenetic representation. For example, the characterizations may be based on: (i) a number of occurrences of a portion of information elements in the perturbated outputs, (ii) a relative frequency of occurrences of a first portion of information elements with respect to occurrences of a second portion of information elements in the perturbated outputs, and/or (iii) other bases for statistical characterization.

The variability information may provide insight into decision-making by the generative AI model and/or information usable to assess usability of the outputs (e.g., information regarding factors affecting stability of the outputs); therefore, the variability information may be provided to downstream consumers 106 as a portion of the computer-implemented services. To improve analysis of the variability information by downstream consumers 106, the variability information may be provided to downstream consumers 106 via a GUI populated using the phylogenetic representation to facilitate visual interpretation of the variability information.

Thus, embodiments disclosed herein may improve processes of explaining outputs generated by generative AI models to downstream consumers of the outputs using variability information. By comparing perturbated outputs of the generative AI models using a structured representation for information elements of the perturbated outputs, the variability information may be obtained and provided to downstream consumers 106 to improve usability of the outputs.

To provide the above noted functionality, the system of FIG. 1 may include data processing systems 100, AI model manager 102, downstream consumers 106, and communication system 104. Each of these components is discussed below.

Data processing systems 100 may provide all, or a portion of, the computer-implemented services. Data processing systems 100 may include any number of data processing systems (e.g., 100A-100N) that may provide generative AI related services to downstream consumers 106.

For example, downstream consumer 106A may subscribe to output generation services provided by data processing system 100A and downstream consumer 106A may provide prompts to data processing system 100A as part of the subscribed services. In response to the prompts, data processing system 100A may provide inference generation services of the computer-implemented services by providing outputs to downstream consumer 106A, and explainability services of the computer-implemented services by providing information usable to assess stability (e.g., reliability, usability) of the outputs.

To provide computer-implemented services (e.g., inference generation services), data processing systems 100 may: (i) obtain prompts for generative AI models (e.g., hosted and/or operated by data processing systems 100), (ii) feed the prompts into the generative AI model to obtain outputs, (iii) provide the outputs to downstream consumers 106, and/or (iv) perform other actions.

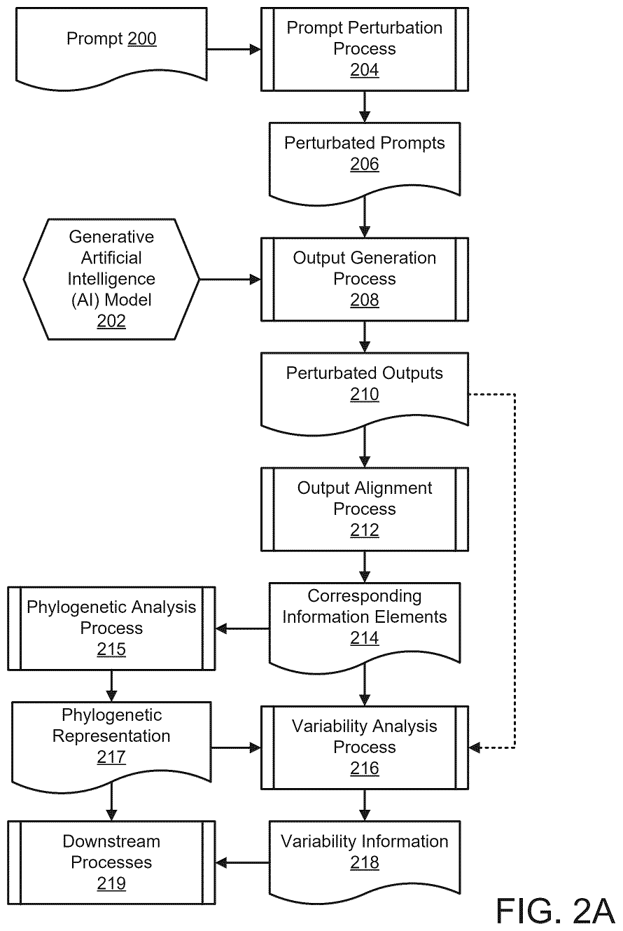

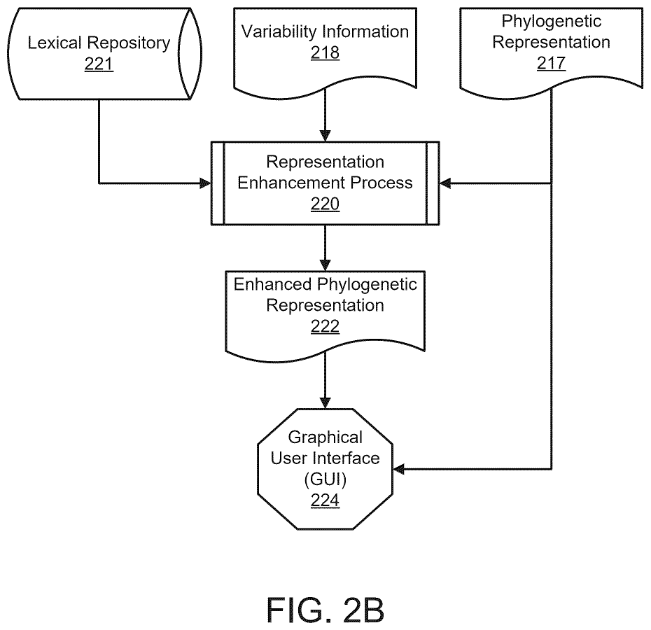

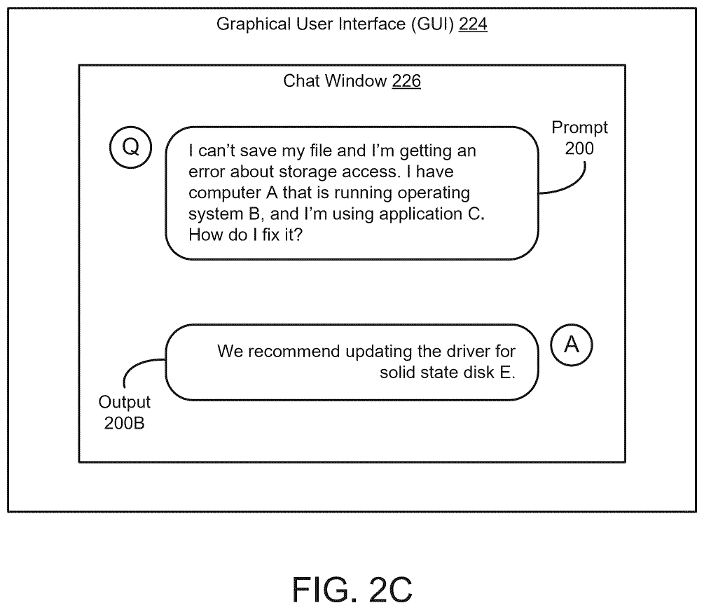

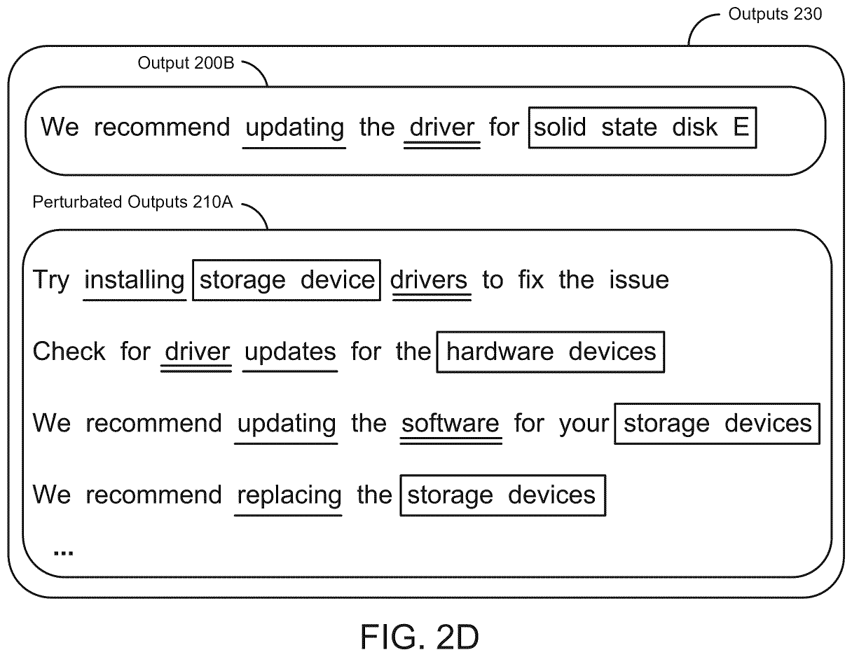

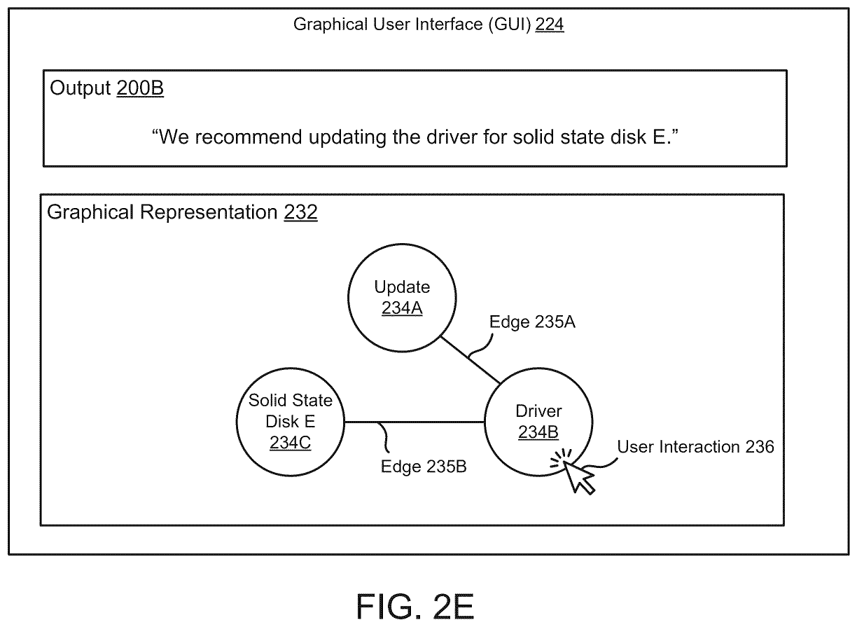

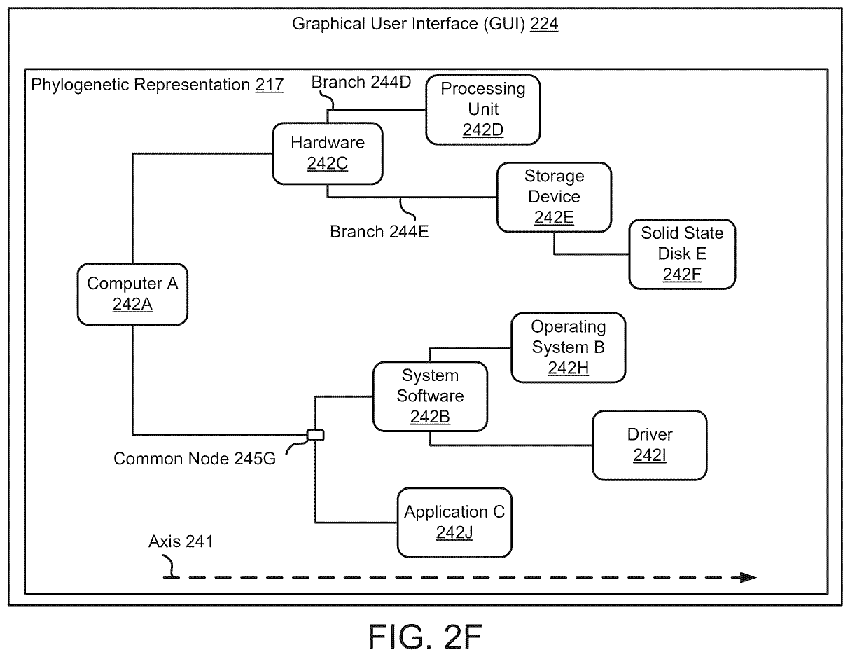

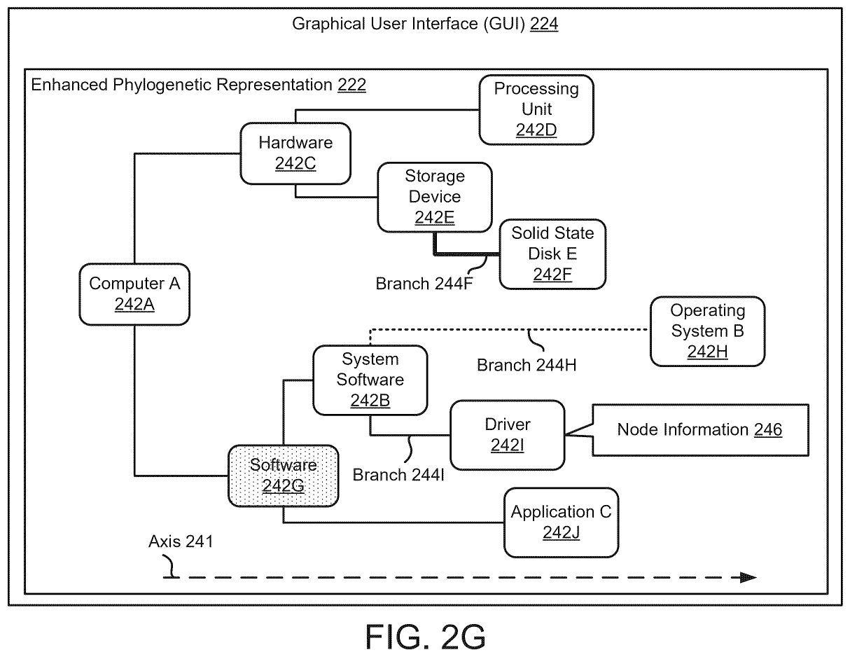

To provide computer-implemented services (e.g., the explainability services), data processing systems 100 may: (i) iteratively perturbate a prompt to obtain perturbated prompts, (ii) feed the perturbated prompts into a generative AI model to obtain perturbated outputs, (iii) identify sets of corresponding information elements of the perturbated outputs, (iv) obtain phylogenetic representations for the sets of corresponding information elements, (v) obtain variability information for elements of the phylogenetic representations, and/or (vi) perform other actions. Refer to FIG. 2A for additional details regarding obtaining variability information for elements of phylogenetic representations.

Data processing systems 100 may provide computer-implemented services to downstream consumers 106 using at least the variability information. For example, data processing systems 100 may provide a phylogenetic representation and variability information for elements of the phylogenetic representation to a downstream process. During the downstream process, the phylogenetic representation may be enhanced using the variability information and/or data from other sources. For example, the enhanced phylogenetic representation may be annotated using portions of the variability information and presented to a downstream consumer via a GUI as a phylogenetic tree and/or a phylogram. Refer to FIG. 2B for additional information regarding enhancing phylogenetic representations. Refer to FIGS. 2D-2G for examples of providing computer-implemented services using variability information.

AI model manager 102 may manage any number of AI models. For example, AI model manager 102 may: (i) obtain training data for AI models, (ii) train any number of AI models to obtain trained AI models, (iii) deploy trained AI models to other entities (e.g., data processing systems 100), (iv) update and/or modify trained AI models (e.g., fine-tune trained AI models based on additional training and/or production data), and/or (v) provide other AI model management services.

For example, AI model manager 102 may cooperatively provide at least a portion of the computer-implemented services to data processing systems 100 by: (i) obtaining training data from downstream consumers 106, (ii) training one or more AI models (e.g., generative AI models) using at least the training data obtained from downstream consumers 106 to obtain one or more trained AI models, (iii) deploying the one or more trained AI models to data processing systems 100 for use in providing output generation services for downstream consumers 106, and/or (iv) performing other actions.

Downstream consumers 106 may provide and/or consume all, or a portion of, the computer-implemented services. Downstream consumers 106 may include any number of downstream consumers (e.g., 106A-106N) and may include, for example, businesses, individuals, and/or devices (e.g., data processing systems). For example, downstream consumers 106 may provide prompts to data processing systems 100 (e.g., from clients, from owners of downstream consumer 106A, from employees of a company that owns downstream consumer 106A). As part of receiving computer-implemented services, downstream consumers 106 may obtain outputs (e.g., in response to the provided prompts) and/or may obtain other information, such as information derived based on the outputs.

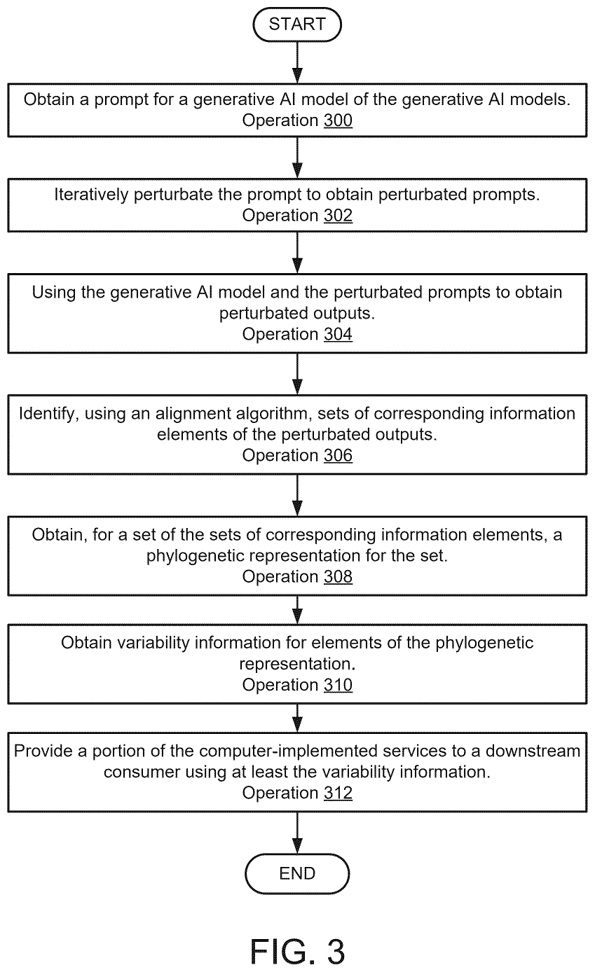

When providing their functionality, any of (and/or components thereof) data processing systems 100, AI model manager 102, and/or downstream consumers 106 may perform all, or a portion, of the actions and methods illustrated in FIGS. 2A-3.

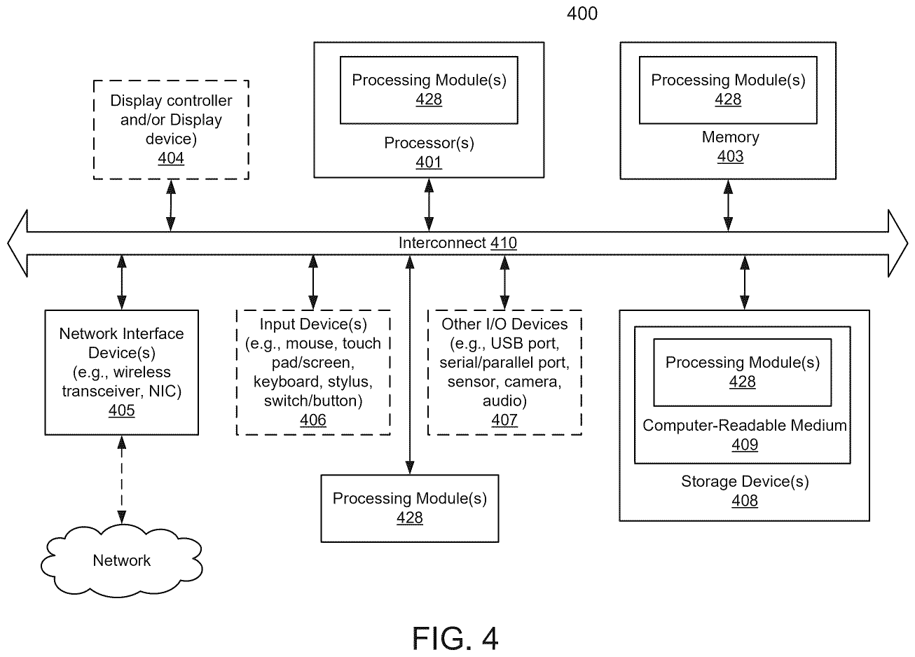

Any of (and/or components thereof) data processing systems 100, AI model manager 102, and downstream consumers 106 may be implemented using a computing device (also referred to as a data processing system) such as a host or a server, a personal computer (e.g., desktops, laptops, and tablets), a “thin” client, a personal digital assistant (PDA), a Web enabled appliance, a mobile phone (e.g., Smartphone), an embedded system, local controllers, an edge node, and/or any other type of data processing device or system. For additional details regarding computing devices, refer to the discussion of FIG. 4.

Any of the components illustrated in FIG. 1 may be operably connected to each other (and/or components not illustrated) with communication system 104. In an embodiment, communication system 104 includes one or more networks that facilitate communication between any number of components. The networks may include wired networks and/or wireless networks (e.g., and/or the Internet). The networks may operate in accordance with any number and types of communication protocols (e.g., such as the internet protocol).

Communication system 104 may allow any of data processing systems 100, AI model manager 102, and downstream consumers 106 to communicate with one another (and/or with other devices not illustrated in FIG. 1). To provide its functionality, communication system 104 may be implemented with one or more wired and/or wireless networks. Any of these networks may be a radio access network (e.g., a cellular core network), a private network (e.g., the “Network” shown in FIG. 4), a public network, and/or may include the Internet. For example, data processing systems 100 may be operably connected to AI model manager 102 and downstream consumers 106 via the Internet. Data processing systems 100, AI model manager 102, downstream consumers 106, and/or communication system 104 may be adapted to perform one or more protocols for communicating via communication system 104.

While illustrated in FIG. 1 as including a limited number of specific components, a system in accordance with an embodiment may include fewer, additional, and/or different components than those illustrated therein.

While providing their functionality, any of data processing systems 100, AI model manager 102, and/or downstream consumers 106 may provide all or a portion of the methods shown in FIGS. 2A-3.

To further clarify embodiments disclosed herein, data flow diagrams in accordance with an embodiment are shown in FIGS. 2A-2B. In these diagrams, flows of data and processing of data are illustrated using different sets of shapes. A first set of shapes (e.g., 200, 206, etc.) is used to represent data structures, a second set of shapes (e.g., 204, 208 etc.) is used to represent processes performed using and/or that generate data, a third set of shapes (e.g., 202) is used to represent generative AI models and/or other types of models, a fourth set of shapes (e.g., 220) is used to represent graphical user interfaces (GUIs), and a fifth set of shapes (e.g., 221) is used to represent data repositories.

Turning to FIG. 2A, a first data flow diagram in accordance with an embodiment is shown. The data flow diagram may illustrate data used in and data processing performed in obtaining variability information for generative AI model outputs.

To obtain the variability information, at least prompt perturbation process 204, output generation process 208, output alignment process 212, phylogenetic analysis process 215, and variability analysis process 216 may be performed.

During prompt perturbation process 204, prompt 200 may be iteratively perturbated to obtain perturbated prompts 206. Prompt 200 may be intended to elicit an output from a generative AI model (e.g., generative AI model 202) that includes information content desired by downstream consumer 106A (e.g., prompt 200 may be obtained from downstream consumer 106A, refer to FIG. 1 for details regarding downstream consumer 106A). Prompt 200, for example, may include human-interpretable text and may include a question. For example, prompt 200 may include: (i) a request from a user for help assessing a malfunction of a computer system, (ii) a request from a customer for a product recommendation, (iii) a request from an employee of a business for a recommendation regarding a financial product (e.g., whether to extend a line of credit to a customer), and/or (iv) other requests. Refer to FIG. 2C for an example of prompt 200.

During prompt perturbation process 204, a first information element of prompt 200 may be modified (e.g., removed, replaced with a replacement information element) to obtain a first perturbated prompt. The first information element may be selected based on: (i) a policy, (ii) an information element replacement schema, (iii) user feedback (e.g., a client may select the information element via a GUI), and/or (iv) via other methods.

The information elements of prompt 200 may include any item, object, or entity in prompt 200 and the information elements may be identified via any algorithm, machine learning model, and/or rule set for natural language processing to extract information from text. Information elements may be extracted from other types of data modalities (e.g., images, audio, video) via object recognition algorithms or models. Information elements may be obtained via other methods without departing from embodiments disclosed herein.

For example, prompt 200 may include the following text: “My computer is not working. It keeps re-starting and overheating. I have computer A and I use operating system B, monitor C, keyboard D, and mouse E. How do I fix it?” The first information elements extracted from prompt 200 may, therefore, include: (i) computer A, (ii) operating system B, (iii) monitor C, (iv) keyboard D, and (v) mouse E.

The information elements may be stored as a list, as a graphical representation of the information elements, as human-interpretable text that summarizes the information elements, and/or in other formats. The information elements (e.g., the human-interpretable text, the graphical representation, the list) may be published to a client portal and may be accessible by a downstream consumer via a GUI. An example of a GUI presenting a graphical representation of information elements is shown in FIG. 2E.

Continuing with the above example, information elements of prompt 200 may be iteratively removed (e.g., one information element may be removed at a time) to generate any number of perturbated prompts (e.g., 206). For example, the information element “operating system B” may be removed from prompt 200 to obtain a perturbated prompt that includes the following text: “My computer is not working. It keeps re-starting and overheating. I have computer A and I use monitor C, keyboard D, and mouse E. How do I fix it?”

Prompt 200 may also be modified, for example, by replacing the first information element with a replacement information element (e.g., provided by a user via a GUI, obtained using a retrieval augmented generation (RAG) process and any number of data sources) to obtain the perturbated prompt. For example, the information element “monitor C” may be replaced with a replacement information element “monitor K” to generate a perturbated prompt that includes the following text: “My computer is not working. It keeps re-starting and overheating. I have computer A and I use operating system B, monitor K, keyboard D, and mouse E. How do I fix it?”

Perturbated prompts 206 and generative AI model 202 may be used to perform output generation process 208. For example, generative AI model 202 may be an LLM (e.g., a neural network inference model) trained to generate language, understand language, and/or otherwise process requests related to languages. Generative AI model 202 may have been previously trained using training data and any training process (e.g., a global optimization process using gradient descent), the training data indicating goals for outputs generated by generative AI model 202 (e.g., outputs). Parameters of generative AI model 202 may be selected using an optimization process (e.g., an objective function may be defined in terms of the training data and outputs generated by generative AI model 202, and a global optimization method such as gradient descent may be used to identify parameters that most faithfully reproduce the trends in the training data). Once the parameters of generative AI model 202 are set, then generative AI model 202 may be used to generate outputs (e.g., responses) based on input data (e.g., prompts). Generative AI model 202 may be trained using other methods without departing from embodiments disclosed herein.

During output generation process 208, perturbated prompts 206 may be provided as ingest to generative AI model 202 and perturbated outputs 210 may be obtained as outputs from generative AI model 202. Output generation process 208 may also include performing retrieval-augmented generation (RAG) processes to augment perturbated prompts 206 with data sources relevant to each of perturbated prompts 206.

Each perturbated output of perturbated outputs 210 may include tokens (e.g., generated by generative AI model 202 during output generation process 208. The tokens may include: (i) words, (ii) letters, (iii) punctuation marks, (iv) phrases, and/or (v) other components of perturbated outputs 210. For example, a first perturbated output may include the following text: “We recommend re-installing driver F and disconnecting and re-connecting all Bluetooth devices.” Tokens of the first perturbated output, therefore, may include: (i) “we,” (ii) “recommend,” (iii) “re-installing,” (iv) “driver F,” (v) “and,” (vi) “disconnecting,” (vii) “re-connecting,” (viii) “all,” and (ix) “Bluetooth devices.” Tokens may include other tokens (e.g., “Bluetooth” and “devices” may be separate tokens, “.” may be an additional token).

Perturbated outputs 210 (e.g., sequences of the tokens) may be aligned to identify corresponding information elements 214. For example, an information element may include one or more tokens. An alignment algorithm may be used to identify corresponding information elements 214 during output alignment process 212.

Output alignment process 212 may implement any type of alignment algorithm for comparing and/or aligning sequences of tokens based on similarities and differences. For example, during output alignment process 212, a first sequence of tokens corresponding to a first perturbated output of perturbated outputs 210 may be obtained. Other sequences of tokens corresponding to other perturbated outputs of perturbated outputs 210 may be aligned relative to the first sequence of tokens using a mathematical formulation (e.g., the other sequences may be shifted to increase, as much as possible, alignment of tokens of the other sequences with tokens of the first sequence). For example, an optimal the alignment of each other sequence of tokens with the first sequence of tokens may be based on some scoring criteria and/or an objective function. A first set of aligned sequences may be obtained based on the optimal alignments of each of the other sequences of tokens with the first sequence of tokens.

During output alignment process 212, corresponding tokens of the first set of aligned sequences may be treated as a first set of corresponding information elements 214. For example, the corresponding tokens may be identified based on values (e.g., alignment scoring values) assigned to each token during alignment, and the first set of corresponding information elements 214 may be selected from the corresponding tokens based on an alignment score threshold. Output alignment process 212 may be performed for the remaining perturbated outputs of perturbated outputs 210 to identify sets of corresponding information elements 214 of perturbated outputs 210.

Corresponding information elements 214 may include any quantity of sets of corresponding information elements. Each set of the sets of corresponding information elements 214 may include subsets of two or more information elements likely to be related in some aspect. For example, the subsets may include: (i) same instances of an information element, (ii) similar information elements (e.g., within a threshold of similarity), and/or (iii) different information elements that correspond to one another based on the alignment of perturbated outputs 210. Refer to FIG. 2C for an example of corresponding information elements identified from a set of aligned sequences.

Corresponding information elements 214 may be provided to subsequent processes to obtain variability information. For example, corresponding information elements 214 may be provided to phylogenetic analysis process 215 and variability analysis process 216. During phylogenetic analysis process 215, a structured representation such as phylogenetic representation 217 may be obtained for the set of corresponding information elements 214.

To obtain phylogenetic representation 217, unique information elements for the set (e.g., each set) of corresponding information elements 214 may be identified. For example, same instances of the information elements may be discarded and/or similar information elements may be combined (e.g., the word “installing” may be combined with the word “install”).

The unique information elements may be provided as input to an algorithm (e.g., software) to establish relationships between the unique information elements, such as semantic relationships (e.g., classifications). The algorithm may estimate the relationships based on differences and/or similarities of characteristics of the unique information elements. The relationships may include, for example, hypernymy or hyponymy (e.g., “device” may be established as a hypernym of “tablet” or “smartphone”), synonymy or antonymy (e.g., “floppy disk” and “diskette” may be established as synonyms), and/or other types of relationships.

During phylogenetic analysis process 215, embeddings of the unique information elements may be used to identify the relationships. The embeddings may include mathematical representations of information elements (e.g., words, phrases), such as vectors, and may be obtained using a machine-learning model and/or other methods. The embeddings may be compared to one another to identify the relationships. For example, a vector-search algorithm may be implemented and/or distances between the embeddings may be used as a metric when a data clustering technique is implemented. Other methods may be used to identify and/or measure relationships between the information elements.

For example, phylogenetic analysis process 215 may implement any type and/or number of computational phylogenetic methods for estimating phylogenies (e.g., relationships) such as Bayesian phylogenetic inference, maximum likelihood, distance matrix, and/or other methods. For example, algorithms similar to those implemented in phylogenetic analysis tools such as MrBayes, Phylogenetic Analysis by Maximum Likelihood (PAML), Phylogenetic Analysis Using Parsimony (PAUP), etc. may be used.

While described with respect to computational phylogenetic methods, phylogenetic analysis process 215 may be performed using other methods and/or algorithms. For example, to obtain phylogenetic representation 217, portions of computational phylogenetic algorithms may be modified (e.g., removed, substituted) and/or other algorithms inspired by (e.g., based on) computational phylogenetic algorithms may be used without departing from embodiments disclosed herein.

Phylogenetic analysis process 215 may be performed for any number of sets of corresponding information elements 214 to obtain phylogenetic representation 217.

Phylogenetic representation 217 may be a structured representation of information elements (e.g., the unique information elements) and their established (e.g., estimated) relationships. Phylogenetic representation 217 may include: (i) elements (e.g., information elements), (ii) information regarding relationships between the elements (e.g., relationship types), and/or (iii) other information usable to represent the elements and their corresponding relationships in the form of an acyclic graph, such as a tree (e.g., a phylogenetic tree).

For example, a phylogenetic tree may include nodes representing a portion of information elements of perturbated outputs 210, and the nodes may be connected by branches indicating a type of relationship between the nodes (e.g., the portion of information elements). An example of a phylogenetic tree is provided in FIG. 2F.

The elements of phylogenetic representation 217 may include a portion of the unique information elements. For example, the elements may include: (i) information elements that are semantically hierarchically related, and/or (ii) information elements that are meaningful with respect to a domain. In other words, in a computing domain, terms like “we,” “recommend,” or “the” may not be included as elements of phylogenetic representation 217, whereas information elements such as “storage” and “solid state disk” may be included as elements of phylogenetic representation 217.

Phylogenetic representation 217 may be provided to variability analysis process 216 to obtain variability information 218 for sets of corresponding information elements 214. Variability information 218 may be based on characterizations for elements of phylogenetic representation 217. For example, variability information 218 may include different measures of variability of instances of an element (e.g., the information element corresponding to the element) present in perturbated outputs 210, and/or aggregate measures of variability of a portion of the elements.

During variability analysis process 216, a characterization for a first element of phylogenetic representation 217 may be obtained. The characterization for the first element may be based on, for example: (i) a number of occurrences of a first information element corresponding to the first element in perturbated outputs 210 (e.g., a count of the occurrences), (ii) a relative frequency of occurrences of the first information element with respect to occurrences of a second information element corresponding to a second element (of the elements of phylogenetic representation 217) in the perturbated outputs, and/or (iv) other bases for characterizing the elements of phylogenetic representation 217.

The number of occurrences of the first element (e.g., a first element count) may be obtained by counting the number of instances of the first information element in perturbated outputs 210. The relative frequency of occurrences of the first information element with respect to occurrences of the second information element may be obtained by dividing the first element count by the second element count (e.g., a count of the number of instances of the second information element in perturbated outputs 210). For example, to obtain the characterizations, variability analysis process 216 may scan perturbated outputs 210 for instances of information elements and/or counts of instances of the information elements in perturbated outputs 210 may be included in corresponding information elements 214.

The characterizations may indicate a level of variability of the elements of phylogenetic representation 217. For example, frequent occurrences of the first information element (e.g., frequently occurring instances of the first information element) in perturbated outputs 210 may indicate low variability of the first information element and/or (at least a portion of) an output that includes the first information element; however, infrequent occurrences of the first information element relative to occurrences of the second information element in perturbated outputs 210 may indicate high variability of the first information element and/or (at least a portion of) the output that includes the first information element. Moreover, an output that includes the second information element may be more likely to be stable than the output that includes the first information element.

Variability analysis process 216 may be performed for any quantity of elements of phylogenetic representation 217 to obtain variability information 218. Variability information 218 may be one or more data structures that include(s): (i) various characterizations (e.g., measures of variability) of portions of perturbated outputs 210 (e.g., with respect to information elements corresponding to elements of phylogenetic representation 217), (ii) information regarding and/or linking variability information 218 to other data structures (e.g., prompt 200, generative AI model 202, perturbated prompts 206, perturbated outputs 210, corresponding information elements 214, phylogenetic representation 217), and/or (iii) other information.

At least variability information 218 and/or phylogenetic representation 217 may be provided to and used by downstream processes 219. Downstream processes 219 may include any type of process for providing computer-implemented services to downstream consumers of outputs of generative AI model 202. For example, downstream processes 219 may include an inferencing process wherein outputs of generative AI model 202 are provided to the downstream consumers, and a management process wherein variability information 218 and/or data derived from variability information 218 is provided to the downstream consumers for use in managing usability of the outputs.

Downstream processes 219 may facilitate services subscribed to by the downstream consumers. The service may include presenting portions of variability information 218 to a downstream consumer in a manner that is easily interpretable by the downstream consumer.

For example, a graphical representation of variability information 218 may be provided to the downstream consumer using a phylogenetic tree and/or an enhanced phylogenetic tree. The trees may be used to provide downstream consumers with a visual, interactive mode of investigation of the variability of portions of perturbated outputs 210. For example, the trees may be used to populate a GUI accessible to (e.g., viewable by) the downstream consumer, and the downstream consumer may interact with the GUI to facilitate an assessment of usability of outputs of generative AI model 202. Refer to FIG. 2C and FIGS. 2E-2G for examples of information that may be displayed to a user via a GUI.

Turning to FIG. 2B, a second data flow diagram in accordance with an embodiment is shown. The second data flow diagram may illustrate data used in and data processing performed to provide computer-implemented services to downstream consumers of generative AI model outputs using variability information. FIG. 2B may illustrate an example of downstream processes 219 of FIG. 2A.

A portion of the computer-implemented services provided to the downstream consumers of the generative AI model outputs may be provided using at least variability information 218. For example, phylogenetic representation 217 and variability information 218 may be provided to representation enhancement process 220 to obtain enhanced phylogenetic representation 222.

During representation enhancement process 220, enhanced phylogenetic representation 222 may be obtained based on phylogenetic representation 217. As discussed with respect to FIG. 2A, phylogenetic representation 217 may include a phylogenetic tree with a plurality of nodes representing at least a portion of information elements of perturbated outputs generated by the generative AI model. The nodes may be connected by branches (e.g., edges) indicating relationships between the information elements, such as hierarchical semantic relationships.

However, portions of the phylogenetic tree may be incomplete. For example, common nodes (e.g., nodes from which two or more branches originate) may be missing due to various reasons, such as an absence of the information elements corresponding to the common nodes in the perturbated outputs and/or limitations of processes used to obtain the phylogenetic tree. Consequently, if the phylogenetic tree used to facilitate model explainability (e.g., via variability of outputs of the model) is incomplete, then the explainability may be limited.

Therefore, to complete an incomplete phylogenetic tree, information from lexical repository 221 may be accessed. Lexical repository 221 may include any type of lexical database. For example, lexical repository 221 may be an organized inventory of words (e.g., morphemes) and corresponding information such as word meanings, part-of-speech designations, sample sentences, cultural annotations, and semantic relationships between words (e.g., lexical databases such as WordNet, Global Lexical Database, Database of Analysed Texts of English (DANTE)).

During representation enhancement process 220, lexical repository 221 may be queried, for example, using elements (e.g., information elements) linked to missing common nodes and/or other elements of the phylogenetic tree to provide context. For example, a natural language processing and/or other computational linguistics techniques may be used to expand a query for lexical repository 221 based on the elements of the phylogenetic tree and/or identify most likely elements to populate missing common nodes (e.g., to impute common nodes). For example, a common node for two nodes representing “keyboard” and “mouse” may be “peripheral” (e.g., a hypernym of both “keyboard” and “mouse”), and “peripheral” may not be a common node for an outgroup to the two nodes.

During representation enhancement process 220, portions of variability information 218 that correspond to elements of phylogenetic representation 217 and/or other information (not shown) may be used to populate metadata of the corresponding elements of enhanced phylogenetic representation 222. As a result, each element (e.g., information element) of enhanced phylogenetic representation 222 may be associated with at least one measure of variability of variability information 218.

Therefore, enhanced phylogenetic representation 222 may be similar to phylogenetic representation 217, and: (i) may include more elements (e.g., populated common nodes) than phylogenetic representation 217, and/or (ii) may include portions of variability information 218 corresponding to its elements.

For example, enhanced phylogenetic representation 222 may be a phylogram including at least one node representing an information element that does not correspond to any of the information elements represented by nodes of the phylogenetic tree (e.g., an imputed node that was populated during representation enhancement process 220). Nodes (and/or branches) of the phylogram may be annotated (e.g., associated with, via corresponding metadata) with measures of variability from variability information 218. An example of a phylogram is provided in FIG. 2G.

Graphical user interface (GUI) 224 may be populated using phylogenetic representation 217 and/or enhanced phylogenetic representation 222 to obtain a populated GUI. GUI 224 may be accessible by downstream consumers (e.g., via a website) and may include any number of pop-up interfaces, chat windows, buttons, graphics, and/or other elements through which information may be displayed to a viewer (e.g., a downstream consumer) and/or through which the viewer may interact. Refer to FIG. 2F and FIG. 2G for an example of a GUI populated using a phylogenetic representation and an enhanced phylogenetic representation, respectively.

Any of the processes illustrated using the second set of shapes may be performed, in part or whole, by digital processors (e.g., central processors, processor cores, etc.) that execute corresponding instructions (e.g., computer code/software). Execution of the instructions may cause the digital processors to initiate performance of the processes. Any portions of the processes may be performed by the digital processors and/or other devices. For example, executing the instructions may cause the digital processors to perform actions that directly contribute to performance of the processes, and/or indirectly contribute to performance of the processes by causing (e.g., initiating) other hardware components to perform actions that directly contribute to the performance of the processes.

Any of the processes illustrated using the second set of shapes may be performed, in part or whole, by special purpose hardware components such as digital signal processors, application specific integrated circuits, programmable gate arrays, graphics processing units, data processing units, and/or other types of hardware components. These special purpose hardware components may include circuitry and/or semiconductor devices adapted to perform the processes. For example, any of the special purpose hardware components may be implemented using complementary metal-oxide semiconductor-based devices (e.g., computer chips).

Any of the data structures illustrated using the first set of shapes may be implemented using any type and number of data structures. Additionally, while described as including particular information, it will be appreciated that any of the data structures may include additional, less, and/or different information from that described above. The informational content of any of the data structures may be divided across any number of data structures, may be integrated with other types of information, and/or may be stored in any location.

Thus, by implementing the data flow shown in FIG. 2A, a system in accordance with embodiments disclosed herein may be used to obtain: (i) perturbated outputs from generative AI models based on perturbated prompts, and (ii) variability information for information elements of the perturbated outputs. By doing so, a measure of variability of portions of the perturbated outputs may indicate a stability of (the portions of) the perturbated outputs, thereby providing a means for assessing reliability of the generative AI model and/or its outputs in a structured manner. Consequently, a usefulness of the outputs may be increased for downstream consumers of the outputs.

By implementing the data flow shown in FIG. 2B, the system may be used to provide computer-implemented services to the downstream consumers by presenting the variability information to the downstream consumers in a visually interpretable manner using a structured representation. Doing so may increase a likelihood of the downstream consumers timely assessment of the variability information with respect to the usefulness of the outputs of the generative AI model.

Turning to FIG. 2C, an example of displaying a prompt and a corresponding output from a generative AI model to a user of a GUI is shown. A user (e.g., downstream consumer 106A described in FIG. 2A) may access GUI 224 through which the user may receive assistance with assessing a malfunction of a computer system. A customer may open chat window 226 as part of functionality of GUI 224 and may enter prompt 200 into the chat window.

Prompt 200 may include the following text: “I can't save my file and I'm getting an error about storage access. I have computer A that is running operating system B, and I'm using application C. How do I fix it?” Prompt 200 is shown next to a circle with the letter “Q” inside the circle to indicate a question from the customer that was submitted via chat window 226.

Output 200B may include the following text: “We recommend updating the driver for solid state disk E.” Output 200B is shown next to a circle with the letter “A” inside the circle to indicate an answer from the manufacturer/vendor for the product (e.g., via the generative AI model). Output 200B may be visible to the user via chat window 226.

To provide variability information to the user, prompt 200 may be iteratively perturbated (e.g., modified) as described in prompt perturbation process 204 in FIG. 2A. The perturbated prompts may be fed into the generative AI model (e.g., 202) to obtain perturbated outputs as described in output generation process 208 in FIG. 2A to obtain sets of aligned sequences, and corresponding information elements may be identified from the sets of aligned sequences.

Turning to FIG. 2D, an example of corresponding information elements of outputs from a generative AI model is shown. Outputs 230 may include output 200B (e.g., generated based on prompt 200), and an example subset of perturbated outputs 210A obtained based on perturbations to prompt 200. As described with respect to FIG. 2A, an alignment process may be performed based on sequences of the tokens of each of outputs 230 to identify corresponding information elements of outputs 230.

In FIG. 2D, examples of corresponding information elements are indicated using single underlining of an information element, double underlining of an information element, and/or a box drawn around an information element. For example, a first set of corresponding information elements is indicated using single underlining (e.g., “updating,” “installing,” “updates,” “updating,” “replacing”), a second set of corresponding information elements is indicated using double underlining (e.g., “driver,” “drivers,” “driver,” “software”), and a third set of corresponding information elements is indicated using boxes drawn around the information elements (e.g., “solid state disk E,” “storage device,” “hardware devices,” “storage devices,” “storage devices”).

The corresponding information elements may be provided to a phylogenetic analysis process (e.g., 215 of FIG. 2A) to obtain a phylogenetic representation for each of the sets, and/or a variability analysis process (e.g., 216 of FIG. 2A) to obtain variability information for elements (e.g., information elements) of the phylogenetic representation. The variability information may be provided to a downstream consumer to facilitate variability analysis of outputs 230. For example, the downstream consumer may navigate the analysis via a GUI displaying a graphical representation of any of outputs 230.

Turning to FIG. 2E, an example of displaying a graphical representation of an output from a generative AI model to a downstream consumer is shown. To obtain the graphical representation, outputs of the generative AI model (e.g., outputs 230 of FIG. 2D) may be provided to a representation generation process (not shown) to obtain corresponding graphical representations. The graphical representation may include a graph, such as an undirected graph, with nodes representing information elements of the input, and edges connecting portions of the nodes representing relationships between the information elements.

The graphical representation may be displayed to the downstream consumer via a GUI. In the example shown in FIG. 2E, output 200B and graphical representation 232 (e.g., of output 200B) are displayed to a user of GUI 224. To obtain graphical representation 232, information elements identified in output 200B may be represented as nodes (e.g., nodes 234A-342C) with edges between the nodes (e.g., edges 235A-235B) indicating relationships between the information elements.

For example, the information elements identified from output 200B may include: (i) “update” represented by node 234A, (ii) “driver” represented by node 234B, and (iii) “solid state disk E” represented by node 234C. The edges (e.g., edge 235A, edge 235B) may represent relationships (e.g., syntactic relationships, operational dependencies, data dependencies, entities that cooperatively perform workloads). For example, edge 235A connects node 234A and node 234B indicating that the driver is what is to be updated, and edge 235B connects node 234B and 234C to indicate that solid state disk E is operationally dependent on the driver.

Descriptive information may be embedded in the nodes as a portion of metadata. For example, information regarding solid state disk E (e.g., a hardware identifier, specifications, firmware versions, data connections) may be included in metadata of node 234C. The metadata may be accessed (e.g., displayed to the downstream consumer) based on a user interaction with GUI 224, such as user interaction 236.

The user may interact with GUI 224 to investigate an information element of interest. For example, user interaction 236 may include left clicking on node 234B to select the corresponding information element (“driver”). Based on the user interaction: (i) descriptive information regarding the selected information element may be displayed to the user, and/or (ii) the user may be presented with an option for viewing a structured representation (e.g., a phylogram) that includes variability information related to the selected information element.

Turning to FIG. 2F, an example of displaying a phylogenetic representation to a downstream consumer is shown. Phylogenetic representation 217 may be generated based on a previous user interaction with GUI 224 (and/or another GUI). For example, a user may wish to investigate usability of an output from a generative AI model with respect to an information element of the output, causing the phylogenetic representation 217 to be generated and/or used to populate a GUI viewable by the user, such as GUI 224. In the example shown in FIG. 2F, phylogenetic representation 217 is rendered as a phylogenetic tree.

The phylogenetic tree may include nodes (e.g., nodes 242A-242F, and nodes 242H-242J) representing information elements (e.g., of outputs of the generative AI model), and branches (e.g., branches 244D-244E) indicating relationships between the information elements. The relationships may include hierarchical semantic relationships. For example, node 242C representing the information element “hardware” is a hypernym (e.g., an umbrella or blanket term) for both information elements “processing unit” and “storage device,” represented by node 242C and node 242E, respectively. Thus, node 242C is shown as a common node of node 242D and node 242E (e.g., sister nodes).

As a portion of interactive investigative tools provided to the user, relationship information between nodes of the phylogenetic tree may be viewable by the user via a user interaction with GUI 224. For example, to view relationship information between node 242C and node 242E, the user may left click on branch 244E. The relationship information may include, for example: (i) any relationships between the nodes (e.g., semantic relationships, other types of relationships), (ii) relationship types, (iii) measures of strength of the relationships (e.g., based on optimization criteria and/or metrics used during a phylogenetic analysis process to estimate the relationships), and/or (iv) other information (e.g., stored in metadata for the nodes).

Portions of relationship information between nodes may be illustrated by the phylogenetic tree. For example, a branch style and/or node style of the phylogenetic tree may be rendered using a rendering schema specifying branch styles and/or node styles based on the relationship information between connected nodes. For example, in FIG. 2F, horizontal axis 241 may denote a distance between vectors representing the information elements of the nodes, increasing in the direction of the arrow. Branch 244D may be rendered using a shorter branch length than that of branch 244E, which may indicate that “processing unit” is more closely related to “hardware” than “storage device” is related to “hardware” (e.g., based on an algorithm used to generate phylogenetic representation 217).

Note that the phylogenetic tree shown in FIG. 2F is missing node information at one branch split (e.g., common node 245G). For example, common node 245G may represent a first hypernym of information elements represented by node 242B, and node 242J. However, the first hypernym may not have been identified (e.g., in outputs of the generative AI model) and/or defined during generation of phylogenetic representation 217.

Note that for illustrative purposes, other branches of the phylogenetic tree are not denoted, and that in practice, the phylogenetic tree may have more nodes and branches than shown in FIG. 2F. In addition, only a portion of the phylogenetic tree may be presented to the user via GUI 224, for example, depending on a (dynamic) preference of the user, and/or depending on an information element of interest of the user. For example, if the information element of interest is “storage device” represented by node 242E, then GUI 224 may show a portion of the phylogenetic tree that includes sister nodes, ancestral nodes (e.g., parent nodes), and descendant nodes (e.g., child nodes) of node 242E (e.g., nodes 242A-242F).

Phylogenetic representation 217 may be enhanced during an enhancement process (e.g., representation enhancement process 220 of FIG. 2B) to obtain an enhanced phylogenetic representation. The enhanced phylogenetic representation may provide downstream consumers of outputs of the generative AI model with additional information usable to investigate (e.g., evaluate, assess) usability of the outputs.

Turning to FIG. 2G, an example of displaying an enhanced phylogenetic representation to a downstream consumer is shown. The enhanced phylogenetic representation may be viewable by the downstream consumer via a GUI (e.g., GUI 224). In the example shown in FIG. 2G, enhanced phylogenetic representation 222 is rendered as a phylogram.

In the example shown in FIG. 2G, the phylogram is rendered based on variability information for each element of the phylogram (e.g., for each information element represented by nodes of the phylogram). For example, the phylogram may be rendered using a rendering schema specifying a branch style (e.g., branch length, line thickness, line style) and/or a node style based on the variability information and/or other information for the corresponding node. The variability information may include any quantity and/or type of measures of variability for different elements (e.g., information elements) of enhanced phylogenetic representation 222.

In the example of FIG. 2G, lengths of branches corresponding to each node (e.g., branch 244H corresponds to node 242H) are rendered based on a first measure of variability of the variability information. The first measure of variability may be user selected and/or may be indicated by horizontal axis 241. Axis 241 may indicate that the first measure of variability is increasing in the direction of the arrow; therefore, longer branches may be associated with more variability than shorter branches.

For example, the first measure of variability may be based on a number of occurrences of each of the information elements in the outputs of the generative AI model. If node 242H is associated with 5 occurrences of “operating system B,” and node 242I is associated with 90 occurrences of “driver,” then the rendering schema may include a branch length function of the first measure of variability (e.g., an inverse function) so that shorter branches lead to nodes associated with lower variability and longer branches lead to nodes associated with higher variability. Note that branch 244I is shorter than branch 244H, indicating that, based on the first measure of variability, that information element “driver” (e.g., of node 242I) is likely to be more stable than information element “operating system B.”

The line style and/or thickness of the branches shown in FIG. 2G may indicate other measures of variability of the variability information. For example, the rendering schema may specify different types of line styles (e.g., branch 244H is rendered with a dashed line, while branch 244I is rendered with a solid line), line thicknesses (e.g., branch 244F is rendered with a thicker line than the line of branch 244I), and/or other styles of display for nodes and/or branches. The branch styles and/or node styles may be based on bucketized ranges of various measures of variability of the variability information.

The rendering schema may specify a color map for branches and/or nodes. The nodes may be colored (e.g., represented using shading in FIG. 2G) based on variability information and/or other information. For example, in FIG. 2G, shaded node 242G denotes a node representing an imputed information element (e.g., imputed during representation enhancement process 220). Note that common node 245G from FIG. 2G is represented in FIG. 2G as node 242G representing information element “software.”

Thus, the phylogram includes one node representing an information element that does not correspond to any of the information elements represented by nodes of the phylogenetic tree of FIG. 2F. The imputed node may provide additional context for interpretation of the phylogram and/or variability information displayed using the phylogram.

While described with respect to an example method of quantifying and illustrating variability in FIG. 2G, it will be appreciated that other methods for quantifying and illustrating variability may be employed without departing from embodiments disclosed herein. For example, the rendering schema may specify other styles of display of enhanced phylogenetic representation 222 and/or variability may be illustrated by distributing (e.g., dividing) measures of variability of ancestral nodes among their descendant nodes.

The user may interact with the phylogram via GUI 224 using menu options or buttons (not shown) in order to: (i) adjust parameters of the rendering schema (e.g., adjust a color map, select various measures of variability), (ii) access (e.g., view) a list of various measures of variability for each node, and/or (iii) interactively adjust a view of the phylogram (e.g., zooming in or out of the phylogram).

For example, the user may select (e.g., left click on) node 242I, and node information 246 may be displayed via GUI 224. Node information 246 may include variability information associated with the selected node (e.g., measures of variability for the information element “driver”), and/or any type of information relating to the information element represented by the selected node. Information displayed as node information 246 may include: (i) aggregated statistics and/or other data derived from the variability information (e.g., an overall variability score based on a function of at least a portion of the variability information and/or a set of thresholds defining bucketized ranges for the variability score), (ii) a Boolean imputation value for the node, and/or (iii) information associated with the definition of the structured representation (e.g., the phylogram) with respect to the node.

The phylogram (and/or the phylogenetic tree of FIG. 2F) may facilitate discrimination of the information elements for an objective of a user of GUI 224. For example, the objective of the user may be to assess a usefulness of an output from the generative AI model based on variability of portions of the output. Returning to the example of FIG. 2C with prompt 200, “I can't save my file and I'm getting an error about storage access. I have computer A that is running operating system B, and I'm using application C. How do I fix it?” the user may assess the usefulness of output 200B, “We recommend updating the driver for solid state disk E,” based on the phylogram.

For example, the user may use the phylogram to: (i) assess the stability of output 200B and/or portions thereof, (ii) confirm reliability (or unreliability) of output 200B, and/or (iii) guide subsequent decision-making processes based on output 200B (e.g., to manually refine output 200B).

For example, the variability of portions of the outputs visualized using on the phylogram may indicate to the user that output 200B is stable based on low variability for information elements “driver” and “solid state disk E.” In other words, the user may confirm based on the phylogram that a driver related to solid state disk E is likely to be the issue. The user may interact with the phylogram (e.g., to plot and/or view different measures of variability) to confirm that it is most likely the device driver for solid state disk E that needs to be updated to fix the storage issue.

Thus, the user may assess usability of output 200B using structured representations of variability of outputs from the generative AI model. By doing so, the user may be more likely to trust and/or rely on at least portions of output 200B for subsequent decision-making processes. As a result, the user may be more likely to obtain desired computer-implemented services using the generative AI model.

As discussed above, the components of FIG. 1 may perform various methods to manage a data processing system. FIG. 3 illustrates methods that may be performed by the components of the system of FIG. 1. In the diagram discussed below and shown in FIG. 3, any of the operations may be repeated, performed in different orders, and/or performed in parallel with or in a partially overlapping in time manner with other operations.

Turning to FIG. 3, a flow diagram illustrating a method of providing computer-implemented services using generative AI models in accordance with an embodiment is shown. The method may be performed, for example, by any of the components of the system of FIG. 1, and/or other components not shown therein.

At operation 300, a prompt may be obtained for a generative AI model of generative AI models. Obtaining the prompt for the generative AI model may include: (i) reading the prompt from storage, (ii) receiving (e.g., via a communication system) the prompt from another entity, (iii) receiving the prompt as part of user feedback submitted via a GUI (e.g., a chat interface displayed on the user's device), and/or (iv) other methods.

At operation 302, the prompt may be iteratively perturbated to obtain perturbated prompts. Iteratively perturbating the prompt may include: (i) during a first perturbation, removing a first information element (e.g., a first token) of the prompt obtain a first perturbated prompt, (ii) during a second perturbation, removing a second information element of the prompt to obtain a second perturbated prompt, and/or (iii) other methods. Iteratively perturbating the prompt may include any number of perturbations to obtain any number of perturbated prompts.

Iteratively perturbating the prompt may also include: (i) replacing the first information element with a first replacement information element to obtain a third perturbated prompt, (ii) replacing the first information element with a second replacement information element to obtain a fourth perturbated prompt, and/or (iii) other methods.

Removing the first information element may include: (i) selecting the first information element (e.g., randomly, based on a schema, based on feedback from a downstream consumer, based on feedback from a subject matter expert), (ii) deleting the first information element from the prompt, (iii) using a model (e.g., a second generative AI model) to create the first perturbated prompt, and/or (iv) other methods.

Replacing the first information element with the replacement information element may include: (i) deleting the first information element from the prompt, (ii) adding the replacement information element to the prompt, (iii) providing the replacement information element and the prompt to another entity responsible for replacing the first information element, and/or (iv) other methods.

If the at least one action performed as part of iteratively perturbating the prompt includes replacing the first information element with a replacement information element, the replacement information element may be obtained prior to replacing the first information element.

Obtaining the replacement information element may include: (i) obtaining an information element replacement schema (e.g., from storage, from another entity via a communication system, from a downstream consumer via a GUI), (ii) reading instructions from the information element replacement schema (e.g., for selecting the first information element, for obtaining the replacement information element), (iii) selecting, based on the information element replacement schema, the first information element, (iv) performing a search process (e.g., a lookup process, a RAG process) using the first information element to obtain one or more replacement information elements (e.g., from a database, from another entity, from any number of other data sources), and/or (v) other methods.

At operation 304, the generative AI model and the perturbated prompts may be used to obtain perturbated outputs. Using the generative AI model and the perturbated prompts to obtain the perturbated outputs may include: (i) feeding perturbated prompts (e.g., as is, following data processing such as a RAG process) into the generative AI model to obtain the plurality of the outputs from an output layer of the generative AI model, (ii) providing the perturbated prompts to another entity responsible for operating the generative AI model, and/or (iii) other methods.

At operation 306, sets of corresponding information elements of the perturbated outputs may be identified using an alignment algorithm. The sets of corresponding information elements may be identified by: (i) receiving a notification (e.g., from another device) indicating the sets of corresponding information elements, (ii) reading a data structure (e.g., from storage) indicating the sets of corresponding information elements, (iii) performing an output alignment process that implements the alignment algorithm to identify the corresponding information elements, and/or (iv) other methods.

For example, identifying the sets of corresponding information elements may include: (i) obtaining a first sequence of tokens corresponding to a first perturbated output of the perturbated outputs, (ii) aligning the first sequence of the tokens to other sequences of tokens corresponding to other perturbated outputs of the perturbated outputs to obtain a first set of aligned sequences, (iii) treating corresponding tokens of the first set of aligned sequences as a first set of the sets of corresponding information elements, and/or (iv) other methods.

Obtaining the first sequence of tokens may include: (i) reading the first sequence of tokens from storage, (ii) receiving the first sequence of tokens from another entity (e.g., via a communication system, via an application programming interface (API)), (iii) generating the first sequence of tokens using a generative AI model (e.g., extracting tokens of the first sequence of the tokens from text using natural language processing and/or other methods), and/or (iv) other methods.

Aligning the first sequence of the tokens to the other sequences of the tokens may include: (i) shifting, using a mathematical formulation, the other sequences relative to the first sequence to increase alignment between tokens of the first sequence and tokens of the other sequences, (ii) providing the first sequence and the other sequences to another entity and receiving the first set of aligned sequences from the entity, and/or (iii) other methods.

Shifting the other sequences relative to the first sequence may include: (i) performing any alignment algorithm (e.g., dynamic programming, progressive alignment construction, iterative methods, consensus methods) using the first sequence and the other sequences, (ii) providing the first sequence and the other sequences to another entity responsible for executing the algorithm, and/or (iii) other methods.

Treating corresponding tokens of the first set of aligned sequences as the first set of the sets of corresponding information elements may include: (i) identifying a set of aligned tokens in the first set of aligned sequences, (ii) providing the set of aligned tokens to a process as the first set of corresponding information elements, (iii) generating a data structure that includes identifiers for each token of the set of aligned tokens, and storing the data structure as the first set of corresponding information elements, and/or (iv) other methods.

At operation 308, for a set of the sets of corresponding information elements, a phylogenetic representation may be obtained. The phylogenetic representation may be obtained by: (i) reading the phylogenetic representation from storage, (ii) receiving the phylogenetic representation from another entity (e.g., via a communication system, via an application programming interface (API)), (iii) generating the phylogenetic representation, and/or (iv) other methods.

Obtaining the phylogenetic representation for the set may include: (i) identifying unique information elements of the set, (ii) obtaining a first embedding of a first unique information element of the unique information elements, (iii) performing a phylogenetic analysis process using at least the first embedding to obtain at least a portion of the phylogenetic representation, and/or (iv) other methods.

The unique information elements may be identified by: (i) obtaining a list of the set of corresponding information elements, (ii) ordering the list (e.g., alphabetically) to obtain a sorted list, (iii) comparing adjacent (e.g., subsequent) entries on the sorted list to identify duplicates (e.g., same and/or similar entries, based on a threshold of similarity), (iv) removing the duplicates from the sorted list, (v) treating the remaining entries in the sorted list as the unique information elements.

Identifying the unique information elements may include: (i) reading a data structure (e.g., from storage) indicating the unique information elements, (ii) receiving a message (e.g., from a device) including the unique information elements, and/or (iii) providing the set of corresponding information elements to an entity responsible for identifying unique information elements, and obtaining the unique information elements as a response from the entity.

The first embedding may be obtained by: (i) reading the embedding from storage, (ii) receiving the embedding from another entity (e.g., via a communication system, via an application programming interface (API)), (iii) generating the embedding, and/or (iv) other methods. For example, the first embedding may be generated by providing the first unique information element and/or other information to a model (e.g., a language model) trained to output at least embeddings based on ingest data.

The phylogenetic analysis process may be performed by providing the first embedding and/or other information (e.g., other embeddings of other unique information elements, optimization criteria, metrics) to an optimization algorithm. For example, the optimization algorithm may include any type of phylogenetics software tools designed to produce phylogenetic representations (e.g., trees) based on input data. The optimization algorithm may output at least the portion of the phylogenetic representation.

Performing the phylogenetic analysis process may include providing the first embedding and/or the other information to an entity responsible for obtaining phylogenetic representations, and receiving at least the portion of the phylogenetic representation from the entity in response.

For example, the portion of the phylogenetic representation (e.g., and other portions of the phylogenetic representation obtained based on other sets of corresponding information elements) may be used (e.g., combined) to obtain a phylogenetic tree. The phylogenetic tree may include nodes representing a portion of information elements of the perturbated outputs, and the nodes may be connected by branches indicating relationships between the portion of information elements.

At operation 310, variability information for elements of the phylogenetic representation may be obtained. The variability information may be obtained by: (i) reading the variability information from storage, (ii) providing the phylogenetic representation to another entity responsible for identifying variability information based on phylogenetic representations and receiving the variability information from the entity in response, (iii) generating the variability information based on characterizations of the elements, and/or (iv) other methods.

Obtaining the variability information for the set may include obtaining a characterization for a first element of the elements of the phylogenetic representation. For example, the characterization may be based on a number of occurrences of a first information element corresponding to the first element in the perturbated outputs (e.g., counting the occurrences of the first information element), and a relative frequency of occurrences of the first information element with respect to occurrences of a second information element corresponding to a second element of the elements of the phylogenetic representation in the perturbated outputs (e.g., dividing the count of the occurrences of the first information element by a count of the occurrences of the second information element). Portions of the variability information may be obtained by aggregating characterizations of the first element, the second element, and/or other elements of the phylogenetic representation (e.g., deriving data based on the characterizations).

An enhanced phylogenetic representation may be obtained based on the phylogenetic representation and using at least portions of the variability information corresponding to each of the nodes. The enhanced phylogenetic representation may be obtained by: (i) reading the enhanced phylogenetic representation from storage, (ii) receiving the enhanced phylogenetic representation from another entity (e.g., via a communication system, via an application programming interface (API)), (iii) generating the enhanced phylogenetic representation, and/or (iv) other methods.

For example, the enhanced phylogenetic representation may be generated by: (i) identifying information gaps of the phylogenetic representation (e.g., identifying missing common nodes of the phylogenetic tree), (ii) querying a lexical repository (e.g., lexical repository 221 of FIG. 2B) using information elements related to the information gaps (e.g., information elements represented by ancestral nodes and/or descendant nodes of the missing common nodes) to impute the common nodes (e.g., information elements thereof), (iii) updating metadata of portions of the phylogenetic representation using the variability information, and/or (iv) other methods.

For example, metadata for nodes and/or branches of the phylogenetic tree (of the phylogenetic representation) may be updated using portions of the variability information corresponding to each of the information elements represented by the nodes. A phylogram (of the enhanced phylogenetic representation) may have at least one node (e.g., an imputed common node) representing an information element that does not correspond to any of the information elements represented by nodes of the phylogenetic tree.

At operation 312, a portion of the computer-implemented services may be provided to a downstream consumer using at least the variability information. The portion of the computer-implemented services may be provided to the downstream consumer by: (i) populating, using the phylogenetic representation and/or the enhanced phylogenetic representation, a GUI to obtain a populated GUI, (ii) making the populated GUI viewable by the downstream consumer (e.g., providing access to the GUI to the downstream consumer), and/or (iii) updating the GUI based on input from a user of the GUI (e.g., the downstream consumer) to facilitate discrimination of portions of the phylogenetic representation and/or portions of the enhanced phylogenetic representation for an objective of the user. For example, the phylogenetic representation and/or the enhanced phylogenetic representation may be rendered based on the variability information (e.g., in accordance with a user selection).

Providing the portion of the computer-implemented services may also include: (i) providing data (e.g., an output from the generative AI model based on the prompt, the perturbated outputs, the variability information, and/or other information) to the downstream consumer and/or another entity, and/or (ii) other methods. For example, the downstream consumer and/or the other entity may use the data to meet an objective of the downstream consumer.

Populating the GUI using the phylogenetic representation and/or the enhanced phylogenetic representation may include: (i) uploading, via an application programming interface (API), the phylogenetic representation and/or the enhanced phylogenetic representation to the GUI, (ii) providing the phylogenetic representation and/or the enhanced phylogenetic representation to another entity responsible for uploading data to the GUI, and/or (iii) other methods.

The GUI may display, to the downstream consumer, at least a portion of the phylogenetic representation (e.g., the phylogenetic tree) and/or the enhanced phylogenetic representation (e.g., the phylogram). The phylogenetic representation and/or the enhanced phylogenetic representation may be rendered using a rendering schema, the rendering schema specifying a branch style and/or a node style based on the variability information, as described with respect to FIG. 2G.

The method may end following operation 312.

Thus, as illustrated above, embodiments disclosed herein may provide systems and methods usable to provide computer-implemented services using generative AI models. By providing outputs generated by the generative AI models along with variability information for information elements of the outputs to downstream consumers in a structured manner (e.g., based on a phylogenetic representation), a likelihood of the output meeting needs (e.g., usability needs) of the downstream consumer may be increased.

Any of the components illustrated in FIGS. 1-2G may be implemented with one or more computing devices. Turning to FIG. 4, a block diagram illustrating an example of a data processing system (e.g., a computing device) in accordance with an embodiment is shown. For example, system 400 may represent any of data processing systems described above performing any of the processes or methods described above. System 400 can include many different components. These components can be implemented as integrated circuits (ICs), portions thereof, discrete electronic devices, or other modules adapted to a circuit board such as a motherboard or add-in card of the computer system, or as components otherwise incorporated within a chassis of the computer system. Note also that system 400 is intended to show a high-level view of many components of the computer system. However, it is to be understood that additional components may be present in certain implementations and furthermore, different arrangement of the components shown may occur in other implementations. System 400 may represent a desktop, a laptop, a tablet, a server, a mobile phone, a media player, a personal digital assistant (PDA), a personal communicator, a gaming device, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute a set (or multiple sets) of instructions to perform any one or more of the methodologies discussed herein.

In one embodiment, system 400 includes processor 401, memory 403, and devices 405-407 via a bus or an interconnect 410. Processor 401 may represent a single processor or multiple processors with a single processor core or multiple processor cores included therein. Processor 401 may represent one or more general-purpose processors such as a microprocessor, a central processing unit (CPU), or the like. More particularly, processor 401 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 401 may also be one or more special-purpose processors such as an application specific integrated circuit (ASIC), a cellular or baseband processor, a field programmable gate array (FPGA), a digital signal processor (DSP), a network processor, a graphics processor, a network processor, a communications processor, a cryptographic processor, a co-processor, an embedded processor, or any other type of logic capable of processing instructions.

Processor 401, which may be a low power multi-core processor socket such as an ultra-low voltage processor, may act as a main processing unit and central hub for communication with the various components of the system. Such processor can be implemented as a system on chip (SoC). Processor 401 is configured to execute instructions for performing the operations discussed herein. System 400 may further include a graphics interface that communicates with optional graphics subsystem 404, which may include a display controller, a graphics processor, and/or a display device.

Processor 401 may communicate with memory 403, which in one embodiment can be implemented via multiple memory devices to provide for a given amount of system memory. Memory 403 may include one or more volatile storage (or memory) devices such as random access memory (RAM), dynamic RAM (DRAM), synchronous DRAM (SDRAM), static RAM (SRAM), or other types of storage devices. Memory 403 may store information including sequences of instructions that are executed by processor 401, or any other device. For example, executable code and/or data of a variety of operating systems, device drivers, firmware (e.g., input output basic system or BIOS), and/or applications can be loaded in memory 403 and executed by processor 401. An operating system can be any kind of operating systems, such as, for example, Windows® operating system from Microsoft®, Mac OS®/iOS® from Apple, Android® from Google®, Linux®, Unix®, or other real-time or embedded operating systems such as VxWorks.

System 400 may further include IO devices such as devices (e.g., 405, 406, 407, 408) including network interface device(s) 405, optional input device(s) 406, and other optional IO device(s) 407. Network interface device(s) 405 may include a wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) 406 may include a mouse, a touch pad, a touch sensitive screen (which may be integrated with a display device of optional graphics subsystem 404), a pointer device such as a stylus, and/or a keyboard (e.g., physical keyboard or a virtual keyboard displayed as part of a touch sensitive screen). For example, input device(s) 406 may include a touch screen controller coupled to a touch screen. The touch screen and touch screen controller can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with the touch screen.

IO devices 407 may include an audio device. An audio device may include a speaker and/or a microphone to facilitate voice-enabled functions, such as voice recognition, voice replication, digital recording, and/or telephony functions. Other IO devices 407 may further include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. IO device(s) 407 may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 410 via a sensor hub (not shown), while other devices such as a keyboard or thermal sensor may be controlled by an embedded controller (not shown), dependent upon the specific configuration or design of system 400.

To provide for persistent storage of information such as data, applications, one or more operating systems and so forth, a mass storage (not shown) may also couple to processor 401. In various embodiments, to enable a thinner and lighter system design as well as to improve system responsiveness, this mass storage may be implemented via a solid state device (SSD). However, in other embodiments, the mass storage may primarily be implemented using a hard disk drive (HDD) with a smaller amount of SSD storage to act as an SSD cache to enable non-volatile storage of context state and other such information during power down events so that a fast power up can occur on re-initiation of system activities. Also a flash device may be coupled to processor 401, e.g., via a serial peripheral interface (SPI). This flash device may provide for non-volatile storage of system software, including a basic input/output software (BIOS) as well as other firmware of the system.

Storage device 408 may include computer-readable storage medium 409 (also known as a machine-readable storage medium or a computer-readable medium) on which is stored one or more sets of instructions or software (e.g., processing module, unit, and/or processing module/unit/logic 428) embodying any one or more of the methodologies or functions described herein. Processing module/unit/logic 428 may represent any of the components described above. Processing module/unit/logic 428 may also reside, completely or at least partially, within memory 403 and/or within processor 401 during execution thereof by system 400, memory 403 and processor 401 also constituting machine-accessible storage media. Processing module/unit/logic 428 may further be transmitted or received over a network via network interface device(s) 405.

Computer-readable storage medium 409 may also be used to store some software functionalities described above persistently. While computer-readable storage medium 409 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the machine and that cause the machine to perform any one or more of the methodologies of embodiments disclosed herein. The term “computer-readable storage medium” shall accordingly be taken to include, but not be limited to, solid-state memories, and optical and magnetic media, or any other non-transitory machine-readable medium.

Processing module/unit/logic 428, components and other features described herein can be implemented as discrete hardware components or integrated in the functionality of hardware components such as ASICS, FPGAs, DSPs or similar devices. In addition, processing module/unit/logic 428 can be implemented as firmware or functional circuitry within hardware devices. Further, processing module/unit/logic 428 can be implemented in any combination hardware devices and software components.

Note that while system 400 is illustrated with various components of a data processing system, it is not intended to represent any particular architecture or manner of interconnecting the components; as such details are not germane to embodiments disclosed herein. It will also be appreciated that network computers, handheld computers, mobile phones, servers, and/or other data processing systems which have fewer components or perhaps more components may also be used with embodiments disclosed herein.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Embodiments disclosed herein also relate to an apparatus for performing the operations herein. Such a computer program is stored in a non-transitory computer readable medium. A non-transitory machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

The processes or methods depicted in the preceding figures may be performed by processing logic that comprises hardware (e.g., circuitry, dedicated logic, etc.), software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

Embodiments disclosed herein are not described with reference to any particular programming language. It will be appreciated that a variety of programming languages may be used to implement the teachings of embodiments disclosed herein.

In the foregoing specification, embodiments have been described with reference to specific exemplary embodiments thereof. It will be evident that various modifications may be made thereto without departing from the broader spirit and scope of the embodiments disclosed herein as set forth in the following claims. The specification and drawings are, accordingly, to be regarded in an illustrative sense rather than a restrictive sense.

What is claimed is:

1. A method for providing computer-implemented services using generative artificial intelligence (AI) models, the method comprising:
obtaining a prompt for a generative AI model of the generative AI models;
iteratively perturbating the prompt to obtain perturbated prompts;
using the generative AI model and the perturbated prompts to obtain perturbated outputs;
identifying, using an alignment algorithm, sets of corresponding information elements of the perturbated outputs;
obtaining, for a set of the sets of corresponding information elements, a phylogenetic representation for the set;
obtaining variability information for elements of the phylogenetic representation; and
providing a portion of the computer-implemented services to a downstream consumer using at least the variability information.

2. The method of claim 1, wherein identifying the sets of corresponding information elements comprises:
for a first perturbated output of the perturbated outputs:
obtaining a first sequence of tokens corresponding to the first perturbated output,
aligning the first sequence of tokens to other sequences of tokens corresponding to other perturbated outputs of the perturbated outputs to obtain a first set of aligned sequences, and
treating corresponding tokens of the first set of aligned sequences as a first set of the sets of corresponding information elements.

3. The method of claim 2, wherein aligning the first sequence of the tokens comprises:
shifting, using a mathematical formulation, the other sequences relative to the first sequence to increase alignment between tokens of the first sequence and tokens of the other sequences.

4. The method of claim 3, wherein a token of the tokens comprises at least one type of token selected from a list of types of tokens consisting of:
a word;
a letter;
a punctuation mark; and
a phrase.

5. The method of claim 1, wherein obtaining the variability information for the set comprises:
obtaining a characterization for a first element of the elements of the phylogenetic representation, the characterization being based on at least one selected from a list consisting of:
a number of occurrences of a first information element corresponding to the first element in the perturbated outputs, and
a relative frequency of occurrences of the first information element with respect to occurrences of a second information element corresponding to a second element of the elements of the phylogenetic representation in the perturbated outputs.

6. The method of claim 1, wherein obtaining the phylogenetic representation for the set comprises:
identifying unique information elements of the set;
obtaining a first embedding of a first unique information element of the unique information elements; and
performing a phylogenetic analysis process using at least the first embedding to obtain at least a portion of the phylogenetic representation.

7. The method of claim 6, wherein the phylogenetic representation comprises a phylogenetic tree, the phylogenetic tree comprising nodes representing a portion of information elements of the perturbated outputs, and the nodes being connected by branches indicating relationships between the portion of information elements.

8. The method of claim 7, further comprising:
obtaining an enhanced phylogenetic representation based on the phylogenetic representation and using at least portions of the variability information corresponding to each of the nodes.

9. The method of claim 8, wherein the enhanced phylogenetic representation comprises a phylogram, the phylogram comprising at least one node representing an information element that does not correspond to any of the information elements represented by nodes of the phylogenetic tree.

10. The method of claim 8, wherein providing the portion of the computer-implemented services comprises:
populating, using the phylogenetic representation and/or the enhanced phylogenetic representation, a graphical user interface (GUI) to obtain a populated GUI, the
populated GUI being viewable by the downstream consumer, and the phylogenetic representation and/or the enhanced phylogenetic representation being rendered based on the variability information to facilitate discrimination of portions of the phylogenetic representation and/or portions of the enhanced phylogenetic representation for an objective of a user of the populated GUI.

11. The method of claim 10, wherein the phylogenetic representation and/or the enhanced phylogenetic representation is rendered using a rendering schema, the rendering schema specifying a branch style and/or a node style based on the variability information.

12. The method of claim 1, wherein the generative AI model is a large language model (LLM).

13. A non-transitory machine-readable medium having instructions stored therein, which when executed by a processor, cause the processor to perform operations for providing computer-implemented services using generative artificial intelligence (AI) models, the operations comprising:
obtaining a prompt for a generative AI model of the generative AI models;
iteratively perturbating the prompt to obtain perturbated prompts;
using the generative AI model and the perturbated prompts to obtain perturbated outputs;
obtaining, for a set of the sets of corresponding information elements, a phylogenetic representation for the set;
obtaining variability information for elements of the phylogenetic representation; and
providing a portion of the computer-implemented services to a downstream consumer using at least the variability information.

14. The non-transitory machine-readable medium of claim 13, wherein identifying the sets of corresponding information elements comprises:
for a first perturbated output of the perturbated outputs:
obtaining a first sequence of tokens corresponding to the first perturbated output,
aligning the first sequence of tokens to other sequences of tokens corresponding to other perturbated outputs of the perturbated outputs to obtain a first set of aligned sequences, and
treating corresponding tokens of the first set of aligned sequences as a first set of the sets of corresponding information elements.

15. The non-transitory machine-readable medium of claim 14, wherein aligning the first sequence of the tokens comprises:
shifting, using a mathematical formulation, the other sequences relative to the first sequence to increase alignment between tokens of the first sequence and tokens of the other sequences.

16. The non-transitory machine-readable medium of claim 15, wherein a token of the tokens comprises at least one type of token selected from a list of types of tokens consisting of:
a word;
a letter;
a punctuation mark; and
a phrase.

17. A data processing system, comprising:
a processor; and
a memory coupled to the processor to store instructions, which when executed by the processor, cause operations for providing computer-implemented services using generative artificial intelligence (AI) models to be performed, the operations comprising:
obtaining a prompt for a generative AI model of the generative AI models,
iteratively perturbating the prompt to obtain perturbated prompts,
using the generative AI model and the perturbated prompts to obtain perturbated outputs,
identifying, using an alignment algorithm, sets of corresponding information elements of the perturbated outputs,
obtaining, for a set of the sets of corresponding information elements, a phylogenetic representation for the set,
obtaining variability information for elements of the phylogenetic representation, and
providing a portion of the computer-implemented services to a downstream consumer using at least the variability information.

18. The data processing system of claim 17, wherein identifying the sets of corresponding information elements comprises:
for a first perturbated output of the perturbated outputs:
obtaining a first sequence of tokens corresponding to the first perturbated output,
aligning the first sequence of tokens to other sequences of tokens corresponding to other perturbated outputs of the perturbated outputs to obtain a first set of aligned sequences, and
treating corresponding tokens of the first set of aligned sequences as a first set of the sets of corresponding information elements.

19. The data processing system of claim 18, wherein aligning the first sequence of the tokens comprises:
shifting, using a mathematical formulation, the other sequences relative to the first sequence to increase alignment between tokens of the first sequence and tokens of the other sequences.

20. The data processing system of claim 19, wherein a token of the tokens comprises at least one type of token selected from a list of types of tokens consisting of:
a word;
a letter;
a punctuation mark; and
a phrase.
